(19) United States
(12) Patent Application Publication
(10) Pub. No.: US 2026/0215937 A1
(43) Pub. Date: Jul. 30, 2026

(54) METHOD FOR CONTROLLING A MOVEMENT BEHAVIOUR OF AN ARTIFICIAL JOINT

(71) Applicant: OTTO BOCK HEALTHCARE PRODUCTS GMBH, Wien (AT)

(72) Inventor: Roland AUBERGER, Wien (AT)

(21) Appl. No.: 19/142,179

(22) PCT Filed: Dec. 21, 2023
(86) PCT No.: PCT/EP2023/087221

§ 371 Date: Jun. 20, 2025

(30) Foreign Application Priority Data
Dec. 21, 2022 (DE) 10 2022 134 381.2

(51) Int. Cl. A61F 2/70; A61F 2/64

(52) U.S. Cl. CPC A61F 2/70; A61F 2/64; A61F 2002/701; A61F 2002/704

(57) ABSTRACT

The invention relates to a method for controlling a movement behaviour of an artificial joint which has an upper part and a lower part, mounted thereon to pivot about a pivot axis, between which upper and lower part a device for influencing the pivotability or pivoting of the upper part relative to the lower part is arranged, which device is coupled to a control unit in which a rule set is stored and which activates, deactivates or modulates the device on the basis of input values for the rule set in order to influence the pivoting or pivotability, wherein sensor values of at least one sensor arranged on the upper part or lower part, which are detected during the use of the artificial joint, are supplied to at least one machine-learning-based estimating method which continuously calculates an estimate value for a kinetic or kinematic parameter or an expected kinetic or kinematic parameter from the sensor values and this estimate value is supplied to the rule set as an input value and is used therein as a criterion for activation, deactivation or modulation.

The invention relates to a method for controlling a movement behavior of an artificial joint, in particular an artificial knee joint, which has an upper part and a lower part mounted on the upper part so as to pivot about a pivot axis, between which upper part and lower part a device for influencing the pivotability or pivoting of the upper part relative to the lower part is arranged, which device is coupled to a control device in which a rule set is stored and which activates, deactivates, or modulates the device on the basis of input values for the rule set in order to influence the pivoting or pivotability. Methods and control parameters for controlling the artificial joint are stored in the rule set.

Artificial joints, in particular artificial knee joints, are arranged in prostheses and ortheses, wherein prostheses replace missing limbs in terms of their function and, if applicable, also in terms of their outward appearance. Ortheses are placed on limbs and are used to guide, limit, and, if applicable, influence a movement of a natural limb. Ortheses have orthosis joints, which are arranged or formed between an upper part and a lower part. The upper part and the lower part each have attachment devices for attaching the orthosis to the limb. Attachment devices are arranged on prostheses, with which the prosthesis can be fixed to the limb stump or to the patient. Appropriate devices such as dampers or drives are arranged between the upper part and the lower part of an artificial joint for influencing the pivoting movement or the pivotability, which devices are coupled to a control device via which the dampers or drives are activated, deactivated, or modified in terms of their behavior. Dampers can be designed, for example, as pure passive devices such as linear hydraulics, rotation hydraulics, or magnetorheological dampers. Mechanical brakes can also influence the pivotability or the pivoting movement of the upper part relative to the lower part. Drives are in particular considered to be electric motors and other energy stores, which can prompt or assist a movement or even counteract a movement in order to decelerate a pivoting movement. By using an appropriate circuit, it is also possible to effect a locking of the joint by means of drives and thus prevent pivoting.

The control device activates, deactivates, or modulates the device for influencing the pivoting or pivotability, for example, on the basis of sensor data which are transmitted to the control device. Sensors are arranged on the artificial joint or on the attachment parts such as the prosthesis socket, distal prosthesis components, or orthosis rails. The sensors can also be arranged on the limb of the treated side or on the contralateral side.

To control the change in resistances, for example, state machines stored in the control device are used. A rule set can include multiple state machines which are dynamically activated depending on the situation. The state in which the prosthesis or orthosis is in and how an adjusting device, for example, for a valve, must be activated or deactivated in order to generate a certain movement behavior is derived on the basis of the sensor data. In a hydraulic resistance device, for example, valves are entirely or partially closed in order to change a flow connection in terms of the cross-section in order to influence the corresponding movement of a joint. A control means for a prosthetic knee joint having a state machine is described in EP 549 855 B1.

DE 10 2020 111 535 A1 describes a method for controlling at least one actuator of an orthopedic device with an electronic control device. The control device is coupled to the actuator and at least one sensor and has at least one electronic processor for processing sensor data. At least one state machine in which states of the orthopedic device and state transitions of the actuator are determined is stored in the control device. In addition, a classifier in which sensor data and/or states are automatically classified within the scope of a classification method is stored in the control device.

The classification method and the state machine can be used in combination. On the basis of the classification and the states, a decision is made about the manner of activating or deactivating the actuator as a control signal.

CN 113520683 A describes a control system for a lower limb prosthesis and a method for controlling the prosthesis, in which the prosthesis has a prosthetic knee joint control motor, an ankle joint control motor, a connecting rod, and a shell. An inertial measurement unit (IMU) is used to acquire gait information about a healthy human body under different conditions and, on the basis of the gait information, a training data set is formed. A neural network model is built and trained on the acquired data in a simulation environment. The conventional neural network model is implemented in a control device in a lower limb prosthesis. When the control device receives an input signal from the IMU, an action instruction is output to a joint of the lower limb while taking account of the trained network model.

The article “Knee Angle Estimation Based on IMU Data and Artificial Neural Networks,” Bennett et al., 29th Southern Biomedical Engineering Conference, 2013, pages 111 and 112, describes that the measurement of the knee angle is important for the evaluation of the gait. The measurement can be carried out using an IMU, wherein, due to the fact that the measurement is not direct, the parameters such as knee angle, gait phase, and symmetry of stance can only be estimated. It is examined how an artificial neural network can be used to estimate the knee angle on the basis of acceleration data and data of a gyroscope. It was found that acceleration sensors are the most effective sensors and that the artificial neural network functions best when an IMU is arranged above the knee and an IMU is arranged below the knee.

Angle sensors, for example, knee angle sensors, require installation space at or in the artificial joint and must be mounted, cable-connected, and calibrated separately. The control device requires additional constructive effort in order to be supplied with the data and to process the angle data, wherein the system complexity is increased. The object of the present invention is to provide a method with which it can be sufficiently accurately detected, even without angle sensors, when and how the pivoting or pivotability must be influenced, wherein the least amount of operational effort possible is required.

The object is achieved by a method having the features of the main claim. Advantageous embodiments and developments of the invention are disclosed in the dependent claims, the description, and the figures.

The method for controlling a movement behavior of an artificial joint which has an upper part and a lower part mounted on the upper part so as to pivot about a pivot axis, between which upper part and lower part a device for influencing the pivotability or pivoting of the upper part relative to the lower part is arranged, which device is coupled to a control device in which a rule set is stored and which activates, deactivates, or modulates the device on the basis of input values for the rule set in order to influence the pivoting or pivotability, is distinguished by the fact that sensor values, for example of an IMU arranged on the upper part or the lower part or of IMUs arranged on the upper part and the lower part, which are detected during the use of the artificial joint, are supplied to at least one machine-learning-based estimating method, for example, an artificial neural network, which calculates an estimated value for a kinetic or kinematic parameter, for example, joint angle, or an expected kinetic or kinematic parameter on the basis of the sensor values and this estimated value is supplied to the rule set as an input value, for example, for the joint angle signal, and is used therein as a criterion for the activation, deactivation, or modulation of the device for influencing the pivotability or pivoting. According to one embodiment, multiple identical or different machine-learning-based estimating methods determine multiple estimated values for different kinetic or kinematic parameters and supply these to the rule set. On the basis of the data of the sensor or of the sensors, for example, the IMU or the multiple IMUs, which are arranged on the upper part or the lower part of the artificial joint or the associated components and limbs, the joint angle and, if applicable, another value or other values that can be derived therefrom, are continuously calculated using the machine-learning-based estimating method(s). Instead of positioning a direct angle sensor on the joint, for example, a knee angle sensor, which must be cable-connected and calibrated, in the method according to the invention, a virtual joint angle sensor is generated, which delivers a computed value or an estimated value for the joint angle, in particular the knee angle. The virtual sensor in the machine-learning-based estimating method is based on an evaluation of the data of the sensor or the sensors, in particular of an IMU or multiple IMUs, wherein the machine-learning-based estimating method has been trained, in particular, on the calculation of the joint angle. The method is not limited to artificial joint devices of the lower limb, in particular artificial knee joints or ankle joints, but rather can also be used for other artificial joints, for example, for a hip joint or upper limb joints, for example, an elbow joint, a shoulder joint, or a hand joint.

A machine-learning-based estimating method can be in the form, for example, of an artificial neural network (ANN), for example, a multilayer perceptron, or can include an ANN. Alternatively, black box models and regression methods can be used for this purpose, in which internal model parameters and calculation parameters are optimized in a training process using previously acquired data. A use case for a machine-learning-based estimating method is the estimation of a knee angle on the basis of the data of an IMU mounted on the upper part or the lower part. The training data can be acquired by using a system which includes both a knee angle sensor and an IMU. With these data, the machine-learning-based estimating method is trained to estimate the knee angle on the basis of the IMU data by using the data of the artificial knee angle sensor as “ground truth.” Therefore, the “ground truth” includes a reference result for the training process in that the machine-learning-based estimating method learns to estimate the reference result only on the basis of the available source data, for example, IMU data. If the machine-learning-based estimating method has been trained, it can be used as a virtual sensor for estimating the knee angle. This can replace a physical angle sensor. Alternatively, the machine-learning-based estimating method can also be trained using “unsupervised learning” methods.

Such a calculation or determination of a joint angle or knee angle without a direct joint angle sensor is helpful for stumble recovery in artificial knee joints. In particular when the flexion resistance and the extension resistance can be adjusted separately, stumble recovery can be implemented in artificial knee joints controlled by a control device with a microprocessor, in that the swing phase of the flexion resistance is increased at the point in time of the movement reversal. This point in time is also reliably determined via the computed value or estimated value for the knee angle or the knee angle speed calculated in the machine-learning-based estimating method. In addition, the movement reversal in the swing phase is a unique and specific situation, which is comparatively clearly apparent, and therefore inaccuracies in the calculation or estimation can be easily compensated for or are not particularly important.

The joint angle input value is therefore provided without directly detecting the joint angle using an angle sensor, as a result of which substantially less operational effort and less effort for installation and calibration is required.

The machine-learning-based estimating method continuously determines, in one embodiment, multiple estimated values for one or more kinematic or kinetic parameter(s), so that, for example, not only the joint angle, but also a load, a loading progression, accelerations, the spatial orientation of a component relative to gravity, or the like, can be calculated without the need to directly measure the sought parameter.

In one exemplary embodiment, the sensor values are determined by at least one IMU, for example, in order to estimate the kinetic or kinematic parameter(s) of the joint angle between the upper part and the lower part using the machine-learning-based estimating method(s). Multiple identical or different machine-learning-based estimating methods can also be used to determine multiple estimated values for different kinematic or kinetic parameters, which are then used as the basis for the further procedure. If multiple estimated values for different kinematic or kinetic parameters are present, these are made available to the rule set as input parameters.

In one embodiment, additional sensor data and/or state variables are supplied to the rule set as input values, so that these additional sensor data and/or state variables are used as criteria for activating, deactivating, or modulating the device for influencing the pivotability or the pivoting of the upper part relative to the lower part. This is advantageous specifically in safety-critical cases, since possible inaccuracies of the machine-learning-based estimating method can be compensated for by carrying out a plausibility check on the basis of the additional sensor data. Safety-critical activations, deactivations, or modulations are therefore carried out in one embodiment not exclusively on the basis of the results of the machine-learning-based estimating method, but rather are secured by further parameters, measurements, or calculations.

In order to improve the accuracy of the calculated value and thus the quality of the input variables for the rule set, the machine-learning-based estimating method is supplied and trained with previously acquired sensor data from a database. The previously determined data stored in the database make it easier for the machine-learning-based estimating method to estimate the probability of a certain situation. The database can be continuously updated and coupled to the machine-learning-based estimating method in order to thereby obtain a larger database and greater accuracy.

In one embodiment, the data base is updated during operation in order to achieve optimization in real time. In addition, in order to refine and update the machine-learning-based estimating method, phases of inactivity are provided, for example, during charging or periods of non-use, in which the system can be updated on the basis of additional data. These data can be collected during operation, as a result of which they include patient-specific information. Alternatively, the data can be made available centrally from the manufacturer, for example, via the internet, within the scope of a “field update.”

In one development, the expected joint angle is supplied to the rule set with a lead time between 0.001 seconds and 1 second. It is therefore possible that the control means engages early, but not too early. The machine-learning-based estimating method can make predictions about the assumed behavior of the upper part relative to the lower part and about the development of the current situation, so that it is possible to more quickly respond to possible actual changes. The prediction is thereby, in particular, made possible and becomes more accurate, since, due to the data of the database, the machine-learning-based estimating method can more accurately predict the probability of a future movement or a future movement behavior. Due to these probabilities or estimated values, a corresponding command is transmitted or prepared already via the control device in order to adjust the corresponding behavior, i.e., the pivoting or the pivotability of the upper part relative to the lower part about the pivot axis. On the basis of the sensor values from the IMU, the probabilities of a movement situation or of a state of the joint are calculated in the control device, or the machine-learning-based estimating method, and then supplied to the rule set as input values. The machine-learning-based estimating method thus forms a virtual sensor, which accurately determines a variable, for example, the knee angle, and then transmits this variable to the control device as an input variable. In addition to the knee angle, other variables can also be calculated or predicted, for example, forces, torques, the condition of the ground, or slopes of the ground, so that this variable is also calculated by the machine-learning-based estimating method with the corresponding probability of occurrence and supplied to the control device as an input variable. These input variables are then processed in the particular rule set and act as the basis for changing or influencing the pivoting resistance of the artificial joint.

In one embodiment, sensor values from preceding time increments or periods of time can also be supplied to the machine-learning-based estimating method in order to increase the accuracy for determining the estimated value and, in particular, to also supply the machine-learning-based estimating method with information about the movement history.

The machine-learning-based estimating method or the machine-learning-based estimating methods is/are based, in one embodiment, on Gaussian processes, as a result of which the machine-learning-based estimating method(s) also deliver(s), in addition to the estimated values for the kinematic or kinetic parameter(s), details regarding the confidence interval for the particular estimated value, which are then used in the further procedure. The confidence interval for the particular estimated value is used, for example, as a criterion for activation, deactivation, or modulation and, if applicable, is transmitted together with the respectively determined estimated value and, if applicable, the rule set. This probabilistic approach uses kernal functions and also delivers, in addition to the estimated value, the associated confidence interval, i.e., a statement regarding the quality of the estimate. The additional information is particularly interesting for the control of orthopedic devices, since this enables the weighting of the influence of the estimated values to be established in the rule set for the control for the artificial joint. A small confidence interval corresponds to a good estimation accuracy and thus to a high trustworthiness of the estimated value. D, i.e., the estimated value can be incorporated into the control with a higher weighting than a less reliable estimated value having a high confidence interval.

Estimated values for the information about the condition of the ground and the slope of the ground are advantageous, in particular, for controlling artificial ankle joints or prosthetic feet. For example, the foot position at the end of the swing phase can be modulated according to the estimated slope of the ground. In order to optimize the behavior on steps, an estimated value for the overcome height difference is helpful. Artificial knee joints can modulate the resistance or assistance behavior according to these parameters, for example, offering increased flexion resistance when walking downhill with an increasing descending gradient. When walking downhill, an assist torque can be adapted to the height difference to be overcome. All this additional information required to optimize the assistance by artificial joints can be determined by correspondingly trained machine-learning-based estimating methods as estimated values. Thus, it can be meaningful to implement multiple machine-learning-based estimating methods in the control of an artificial joint, some of which are based on different methods, for estimating different parameters (P). For example, a parameter for the condition of the floor can be estimated using an ANN, whereas, at the same time, a joint angle can be estimated using a method based on Gaussian processes.

Exemplary embodiments of the invention are explained in greater detail in the following with reference to the figures, wherein:

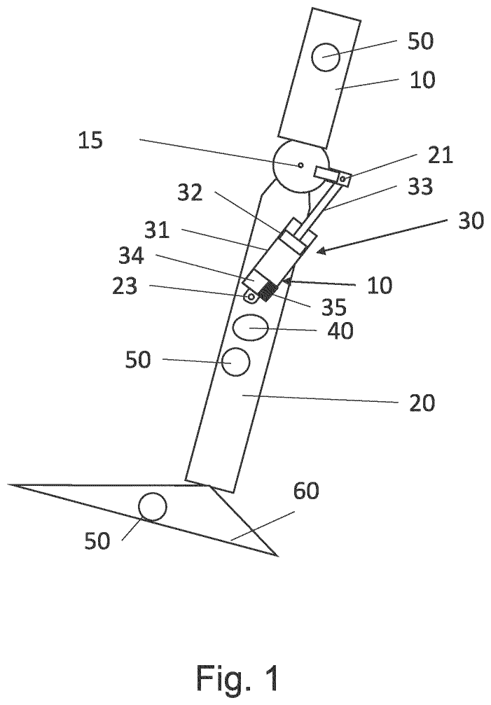

FIG. 1—shows a schematic view of a prosthetic leg;

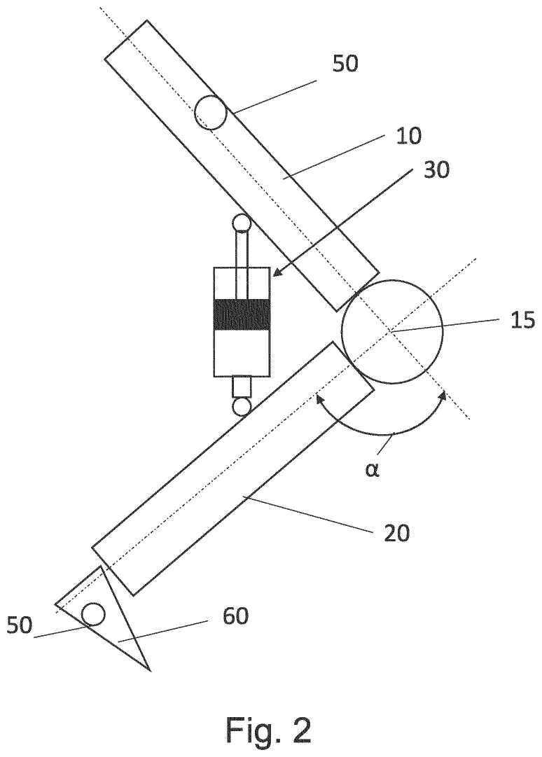

FIG. 2—shows a schematic view of a prosthetic leg in a flexed position;

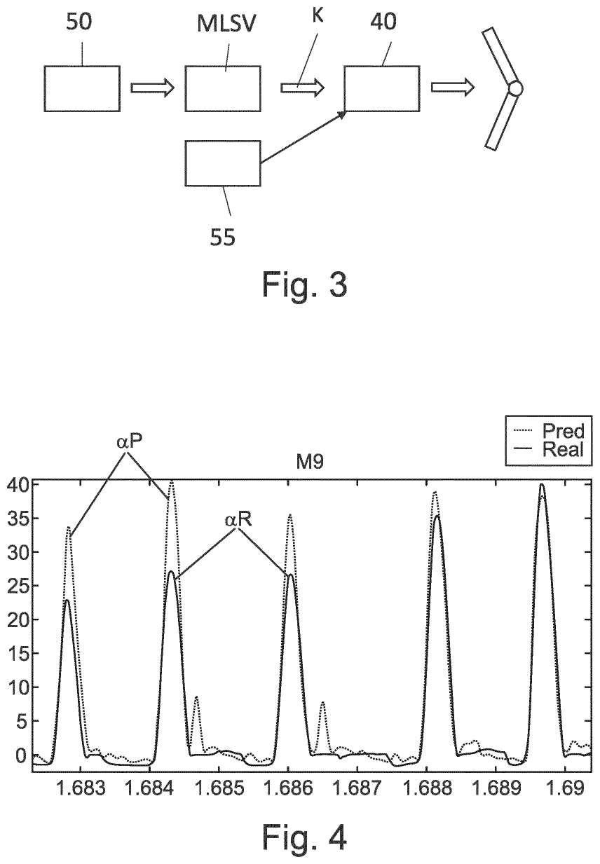

FIG. 3—shows a flowchart;

FIG. 4—shows a diagram of an estimation of a knee angle;

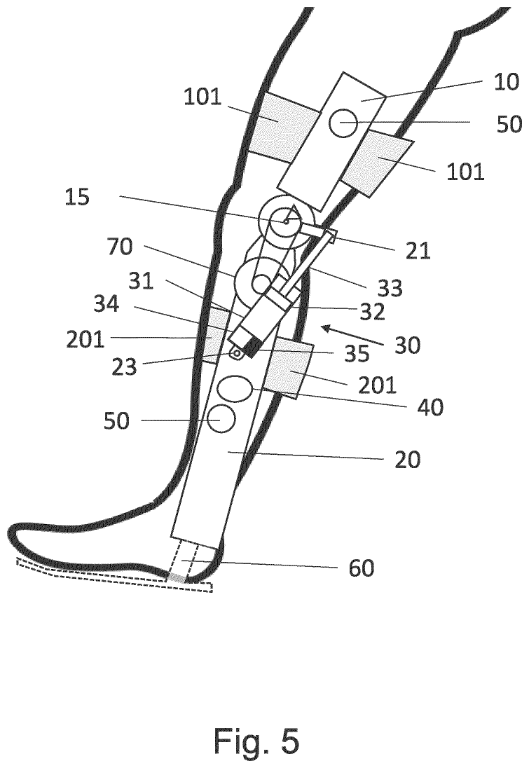

FIG. 5—shows a schematic view of a leg orthosis;

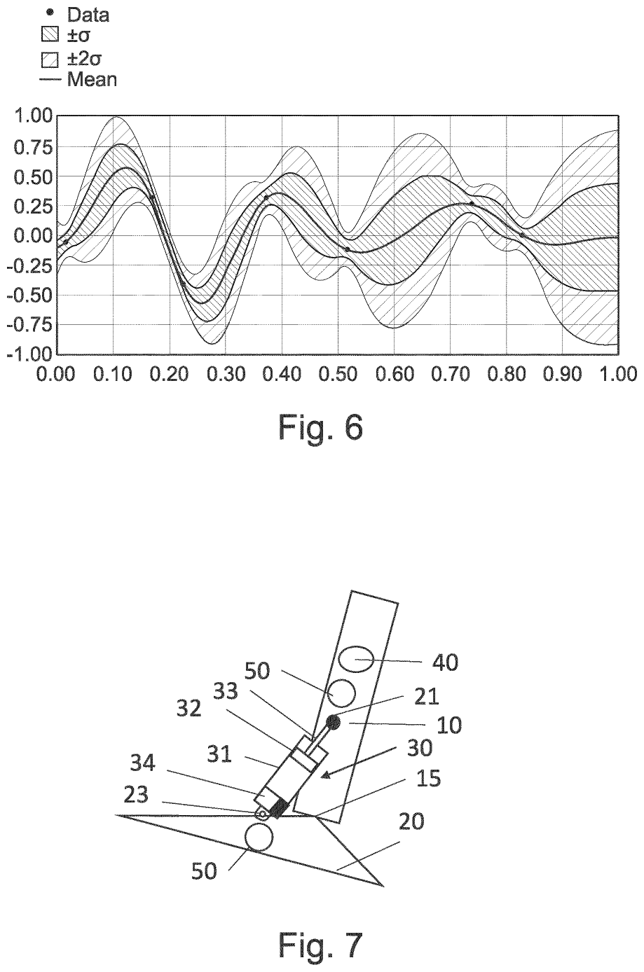

FIG. 6—shows a graph of a regression of a parameter; and

FIG. 7—shows a schematic view of a prosthetic ankle joint.

FIG. 1 shows a schematic view of a prosthetic knee joint as part of a prosthetic leg. The prosthetic knee joint has an upper part 10 and a lower part 20, which are mounted so as to pivot with respect to one another about a pivot axis 15. A prosthetic foot 60 is arranged on the distal end of the lower part 20. In the embodiment as a prosthetic leg according to FIG. 1, a prosthesis socket or another device for accommodating a femoral stump or for attachment to a person is arranged or formed on the upper part 10.

A device 30 for influencing the pivotability or the pivoting of the upper part 10 relative to the lower part 20 is arranged between the upper part 10 and the lower part 20, which device is in the form of a linearly acting hydraulic damper. In the exemplary embodiment shown, the hydraulic damper includes a hydraulic chamber or a cylinder, which is arranged or formed in a housing or the main body 31. A piston 32 is mounted movably in the cylinder. The piston 32 is displaceable along the longitudinal extent of the cylinder and is attached to a piston rod 33, which protrudes from the housing or main body 31. The piston 32 divides the cylinder into chambers which are fluidically interconnected via a hydraulic line. The main body 31 or the housing can be pivotably mounted on the lower part 20 at an attachment point 23 in order to prevent the piston 32 from tilting when the upper part 10 pivots relative to the lower part 20. The end of the piston rod 33 facing away from the piston 32 is attached to the upper part 10 at an upper attachment point 21, in the exemplary embodiment shown to an outrigger in order to increase the distance to the pivot axis 15. During a flexion, the piston 32 is pressed downward, so that the volume of a flexion chamber decreases; correspondingly, the volume of an extension chamber increases, reduced by the volume of the incoming piston rod 33. An electric motor can be arranged in the housing 31 for generating a pressure inside one of the chambers, which electric motor drives a pump (not shown) in order to pressurize the hydraulic fluid inside one of the two chambers and, as a result, move the piston 32 inside the cylinder in one direction or the other. As a result, a flexion movement or an extension movement of the orthopedic device in the form of the prosthetic leg is effected. The electric motor for driving the pump is an option which can be used in combination with the linear damper 30 in one embodiment. In principle, a drive or motor is not necessary for a passive prosthetic knee joint. According to an alternative embodiment of the device 30, instead of a linear damper, in particular linear hydraulics, that which is provided is a rotary damper, in particular, rotation hydraulics, a magnetorheological resistance device, or an electric motor in the generator mode. A combination of several of the aforementioned resistance devices or drives is also implemented in one embodiment. As an alternative to the hydraulic damper, the device 30 can also include a linear actuator, a rotary drive, or a combination of the above-described technologies.

An actuator 34 is arranged inside the housing 31 or on the housing 31, which is coupled to at least one control valve 35 via which the hydraulic resistance in the device 30 can be changed. The actuator 34 is coupled to a control device 40, which activates, deactivates, or modulates the actuator 34 on the basis of sensor values in order to thus be able to provide an adapted resistance and, if applicable, hydraulic locking. In an embodiment of the device 30 as a magnetorheological resistance device, the resistances are changed by activating, deactivating, or modulating a magnetic field, in which case the actuator 34 is the electromagnet or the solenoid. If the device 30 also includes active drives, the control device 40 also delivers commands for active energy output by the drive.

A sensor 50 for detecting the spatial orientation of the lower part 20 and of the upper part 10 is arranged on the upper part 10 and on the lower part 20. In particular, the sensor 50 for detecting the spatial orientation is arranged only on the upper part 10. Via this sensor 50, which is in the form of an IMU (inertial measurement unit), the solid angle or the absolute angle is determined with respect to a fixed spatial orientation, for example, the direction of gravity, during the use of the prosthetic knee joint. In addition to detecting spatial positions, the IMU, as the sensor 50, can also detect other state data, in particular state data relating to the artificial knee joint. The state data that are detected are, in particular, positions, angular positions, velocities, accelerations, forces, and their progressions or changes. The ascertained solid angle of the upper part 10 and/or of the lower part 20 or another state variable is transmitted to the control device 40 as an input variable. The control device 40 modulates, activates, or deactivates the actuator 34 in order to otherwise change the flow resistance in the device 30 in the embodiment as a hydraulic damper, the viscosity, the braking force, or the force counteracting the flexion movement. In order to be able to drive the actuator 34, an energy store is associated therewith, which energy store is in the form of an accumulator. The energy store can be arranged directly next to the actuator 34 or also at another point of the orthopedic device where there is more space available or where this appears advantageous due to the weight distribution. In addition to electromechanical drive units having an accumulator or a battery as an energy store, mechanical energy stores such as springs or flywheels are also provided in embodiments.

Moreover, the control device 40 can be arranged on the prosthesis and at least one further sensor 50 can be arranged on the prosthetic foot 60. All sensors arranged on the prosthesis or the orthosis are coupled to a control device 40 and the sensor values thereof act as a basis for controlling the actuator 34 of the device 30 when the device is in the form of a damper, or as input signals for a motor control when the device 30 is in the form of a motor. For the case of a magnetorheological damping, the sensor values are used to control the magnetic field, or the changing of the magnetic field. On the basis of the sensor data, in particular of the spatial positions, as well as position data and data relating to the loading, orientation, acceleration, and/or deformation of further components, the actuator 34 is activated, for example, to decrease or increase a pivoting resistance, to limit an end stop, and/or to generate or assist a relative motion between the upper part 10 and the lower part 20.

FIG. 2 shows a schematic view of a prosthetic knee joint in a flexed position, having an upper part 10, a lower part 20, and a device 30 between the upper part 10 and the lower part 20 for influencing the pivotability or the pivoting of the component 10 relative to the lower part 20. The upper part 10 can be pivoted relative to the lower part 20 about the pivot axis 15 against the resistance of the device 30 and has an IMU as the sensor 50. In the process, a joint angle α, specifically the knee angle α in the exemplary embodiment shown, between the upper part 10 and the lower part 20 is changed. In the exemplary embodiment shown, the joint angle α is measured on the front side between the upper part 10 and the lower part 20. In a fully extended position, the joint angle α is 0°; as flexion increases, the joint angle α increases and corresponds to the pivot angle. In addition, at least one sensor 50 is arranged on the prosthetic foot 60 on the lower part 20, which sensor is in turn coupled to the control device 40 and detects a load on the foot part 60 or a pivot or position of the foot part 60 in space or relative to the lower part 20.

FIG. 3 shows a flowchart of the control. Sensor values from an IMU as the sensor 50, for example, acceleration values and/or orientations in space from IMUs which are arranged on the upper part 10 and/or the lower part 20 of an orthopedic joint device, for example, a prosthetic knee joint or an orthotic knee joint, are transmitted to a machine-learning-based estimating method. The machine-learning-based estimating method can be trained for regression tasks for estimating output variables or probabilities and for classification tasks. The sensor values from the IMU 50 are evaluated in the machine-learning-based estimating method with respect to a possible knee angle α. The evaluation within the machine-learning-based estimating method yields a computed value or estimated value K, which is processed instead of a direct knee angle sensor signal in the control device 40. The estimated value K acts as an input value or an input variable for the rule set stored in the control device 40. In addition to the estimated value K, further sensor values from sensors 55, which have not been evaluated by the machine-learning-based estimating method, can be supplied to the control device 40 with the rule set. The control device 40 itself determines a control command for the actuator on the basis of the input values in order to increase or decrease a resistance, or to switch on or deactivate a drive. Within the machine-learning-based estimating method, it is possible to calculate a probability that the user of the prosthesis or the orthosis is currently in which situation, or a classification value corresponding to a specific situation. The estimated value K for the knee angle α is thus not measured directly via a knee angle sensor, but rather is preferably determined by the artificial intelligence of the machine-learning-based estimating method on the basis of a sensor value or multiple sensor values from the IMU which is arranged on the upper part 10 or on the lower part 20. To this end, data from a database are used in the artificial intelligence, in an artificial neural network ANN, or another machine-learning-based estimating method to train the artificial intelligence of the machine-learning-based estimating method. In addition to the estimated value K for the knee angle a, further variables or input values for the control device 40, for example, forces or torques which can arise or can be expected due to movement sequences, can be calculated or estimated on the basis of the sensor values from the IMU and, if applicable, from further sensors, which are transmitted to the machine-learning-based estimating method. Due to the evaluation of the sensor data within the machine-learning-based estimating method, it is possible to make predictions or determine probabilities regarding a future progression of variables or characteristic values, since the machine-learning-based estimating method carries out the evaluation on the basis of previous data and data progressions. The use of previous data progressions and the calculation of probabilities makes it possible to observe and anticipate the further progression of the data curve of the variable of the artificial joint with a corresponding probability. However, the longer the period of time to be predicted or anticipated is, the lower is the probability of occurrence of the prediction. Therefore, the lead time with which the machine-learning-based estimating method supplies the control device 40 with the expected joint angle value α or another expected variable is limited. In particular, the limit is between 0.001 seconds and 1 second in order to still have a noticeable effect on the default setting and to implement a change in the setting of the device 30 in a manner that is not too abrupt, which change is not justified by the actual situation in the joint or at the joint. Due to the use of the machine-learning-based estimating method, the use of an estimated value K or computed value which has been determined on the basis of measured course data relating to other orthopedic devices, the future motion sequences and loads can be estimated and used to improve the control. The machine-learning-based estimating method can also be supplied with additional sensor signals deviating from the IMU signals in order to improve the prediction or the computational accuracy for determining the variable to be determined.

By such a method, the control functions without a direct joint angle sensor and the rule set within the control device 40 is supplied exclusively with data from an IMU or multiple IMUs.

FIG. 4 shows the result of an estimation or calculation of the knee angle α using the machine-learning-based estimating method, in the present case an artificial neural network, on the basis of the curve αP predicted by the machine-learning-based estimating method as a dotted line in comparison to a directly measured knee angle αR shown as the solid line. The two curves show substantially the same progression. At the calculated knee angle αP, which has been calculated only on the basis of raw data of the IMU, i.e., accelerations and gyroscope values, deviations from the values αR that are actually present and are directly measured with a knee angle sensor result between the maximum values at small knee angles α. Over the course of the steps, the two curves αP and αR continuously approach one another. In addition, the result can be even further improved due to the incorporation of additional sensor signals, for example, the spatial orientation. The accuracy of the determination via the machine-learning-based estimating method is sufficient, in particular, when determining significant events such as the movement reversal of a lower leg at the end of a swing phase in an orthotic or prosthetic knee joint. The indirect determination without direct knee angle sensors is advantageous in this case, since an additional sensor can be dispensed with.

FIG. 5 shows a schematic view of an orthosis having a basic design that corresponds to that of the prosthesis according to FIG. 1. The connection between the artificial joint and the leg is established in this case via connection devices 101, 201. A difference between the embodiment according to FIG. 1 and the embodiment according to FIG. 5 is that, according to FIG. 5, a drive 70 is additionally provided, in which an electric motor 70 is coupled to a pulley, if applicable via a gear mechanism. A flexion or an extension of the knee joint, depending on the direction of rotation of the motor 70, can then be effected, or assisted, via a V-belt or a toothed belt. The execution with the drive having an electric motor 70 via a mechanical force transmission device and a parallel damping via a hydraulic damper 30 can also be applied in a prosthetic knee joint. As explained above with reference to FIG. 1, in an orthosis, the resistance device can also be in the form of a motor, for example, in the generator mode.

The direct mechanical coupling of the electric motor as a resistance device 30 to the upper part 10 and to the lower part 20 can be established via a force transmission device, for example, via a spindle drive, so that, instead of a piston rod 33, a spindle is extended into or out of the housing 31 by rotating a spindle nut, which is driven by the electric motor. In another embodiment, the motor as the resistance device is coupled to the upper part 10 and to the lower part 20 via a transmission device, for example via a planetary transmission, in order to effect, or decelerate, and influence a displacement of the upper part 10 relative to the lower part 20.

Moreover, the control device 40 and at least one IMU as the sensor 50 are arranged on the prosthesis. The angle between the upper part 10 and the lower part 20 is determined by evaluating the sensor data of two spatial position sensors, or IMUs 50. All sensors arranged on the prosthesis or the orthosis are coupled to a control device 40 and the sensor values from the control device act as a basis for controlling the actuator 34 of the resistance devices 30 when these are in the form of a damper, or as input signals for a motor control for the electric motor 70 when the resistance device 30 is in the form of a motor. For the case of a magnetorheological damping, the sensor values are used to control the magnetic field, or the changing of the magnetic field. On the basis of the sensor data, in particular the spatial positions and/or angular positions as well as position data and data relating to the loading, orientation, acceleration, and/or deformation of further components, the actuator 34 is activated, or the electric motor 70 is activated, deactivated, or modulated, for example, to decrease or increase a pivoting resistance, to limit an end stop, and/or to generate or assist a relative motion between the upper part 10 and the lower part 20.

FIG. 6 shows, by way of example, in the form of the solid line, the result of a regression of a parameter which is plotted on the y-axis, of an input value which is plotted on the x-axis. The ranges ±σ and +2σ plotted about the solid line indicate the respective confidence interval. The input data, which are supplied in the form of training data, are shown as points on the solid line. In the range of the training data, the quality of the estimation is better, the confidence intervals are smaller, indicating that the quality of the calculated data is improved where there is a greater amount of training data.

FIG. 7 schematically shows a prosthetic ankle joint in which the upper part 10 is a lower leg shaft and the lower part 20 is the prosthetic foot. Sensors 50 are arranged on the lower part 20 and on the upper part 10, which are coupled to the control device 40 via which the actuator 30 or the resistance device is correspondingly influenced. For example, the resistance to a dorsal flexion and/or a plantar flexion is adjusted according to an estimated value for a kinetic or kinematic parameter. The estimated value is supplied to the rule set and can also relate, for example, to the slope of the ground.

1. A method for controlling a movement behavior of an artificial joint which has an upper part and a lower part mounted on the upper part so as to pivot about a pivot axis between which upper part and lower part a device for influencing the pivotability or pivoting of the upper part relative to the lower part is arranged, which device is coupled to a control device in which a rule set is stored and which activates, deactivates, or modulates the device on the basis of input values for the rule set in order to influence the pivoting or pivotability, characterized in that sensor values from at least one sensor arranged on the upper part or the lower part (20), which are detected during the use of the artificial joint, are supplied to at least one machine-learning-based estimating method which continuously calculates an estimated value (K) for a kinetic or kinematic parameter (α) or an expected kinetic or kinematic parameter (α) on the basis of the sensor values and this estimated value (K) is supplied to the rule set as an input value and is used therein as a criterion for activation, deactivation, or modulation.

2. The method as claimed in claim 1, characterized in that the machine-learning-based estimating method continuously determines multiple estimated values (K) for multiple kinematic or kinetic parameters (α).

3. The method as claimed in claim 1, characterized in that the sensor values are determined by at least one IMU.

4. The method as claimed in claim 1, characterized in that the kinetic or kinematic parameter (α) includes or represents a joint angle between the upper part and the lower part.

5. The method as claimed in claim 4, characterized in that the joint angle input value is provided without directly detecting the joint angle (α) using an angle sensor.

6. The method as claimed in claim 1, characterized in that additional sensor data and/or state data are supplied to the rule set as input values and used as criteria for the activation, deactivation, or modulation.

7. The method as claimed in claim 6, characterized in that safety-critical activations, deactivations, or modulations are not carried out exclusively on the basis of the results of the machine-learning-based estimating method.

8. The method as claimed in claim 1, characterized in that the machine-learning-based estimating method is supplied and trained with previously acquired sensor data from a database.

9. The method as claimed in claim 8, characterized in that the machine-learning-based estimating method is carried out using a defined data set.

10. The method as claimed in claim 1, characterized in that the machine-learning-based estimating method is updated during operation with current data collected during the operation.

11. The method as claimed in claim 1, characterized in that the machine-learning-based estimating method includes an artificial neural network (ANN).

12. The method as claimed in claim 1, characterized in that the machine-learning-based estimating method is based on a regression method or a parameterized black box model.

13. The method as claimed in claim 1, characterized in that the expected joint angle (α) is supplied to the rule set with a lead time between 0.001 s and 1 s.

14. The method as claimed in claim 1, characterized in that probabilities of a movement situation or of a state of the joint are calculated in the machine-learning-based estimating method on the basis of the sensor values and supplied to the rule set as an input value.

15. The method as claimed in claim 1, characterized in that sensors values from preceding time increments are also supplied to the machine-learning-based estimating method.

16. The method as claimed in claim 1, characterized in that the machine-learning-based estimating method is based on Gaussian processes and delivers statements regarding a confidence interval (KIα) for the particular estimated value (α).

17. The method as claimed in claim 16, characterized in that, in addition to the determined estimated value (α), the associated confidence interval (KIα) is also supplied to the rule set and is used as a criterion for the activation, deactivation, or modulation.

18. The method as claimed in claim 17, characterized in that multiple estimated values (α) are transmitted together with associated confidence intervals (KIα) to the rule set.

19. The method as claimed in claim 1, characterized in that multiple estimated values (α) for different kinematic or kinetic parameters (P) are made available to the rule set as input parameters.
